(19) United States
(12) Patent Application Publication
(10) Pub. No.: US 2023/0225824 A1
(43) Pub. Date: Jul. 20, 2023

(54) PORTABLE AEROSOL-PROTECTIVE APPARATUS FOR USE IN A HOSPITAL OR MEDICAL SETTING

(71) Applicant: United States of Government as Represented by the Department of Veterans Affairs, Washington, DC (US)

(72) Inventor: Sam S. Fares, Brooklyn, NY (US)

(21) Appl. No.: 18/007,622

(22) PCT Filed: Jun. 2, 2021
(86) PCT No.: PCT/US2021/035435

§ 371 Date: Dec. 1, 2022

Related U.S. Application Data
(60) Provisional application No. 63/034,123, filed on Jun. 3, 2020.
(60) Provisional application No. 63/153,661, filed on Feb. 25, 2021.

(51) Int. Cl. A61B 90/40; A61G 10/00

(52) U.S. Cl. CPC A61B 90/40; A61G 10/005; A61B 2560/0437; A61B 2090/401

(57) ABSTRACT

Disclosed herein is a portable aerosol-protective apparatus and method for reducing the exposure of healthcare workers to contaminants and aerosols that may contain respiratory and other pathogens. The apparatus generally comprises a shield suitable for forming a partial enclosure around the head-space end of a hospital or surgery bed, through which a medical procedure can be performed on a patient. The method generally comprises performing the medical procedure on the patient through the shield of the disclosed apparatus.

### CROSS-REFERENCE TO RELATED APPLICATIONS

This application claims priority to and the benefit of the filing date of U.S. Provisional Patent Application No. 63/034,123, filed Jun. 3, 2020, and U.S. Provisional Patent Application No. 63/153,661, filed Feb. 25, 2021. The entirety of each of these applications is hereby incorporated by reference herein.

### FIELD

This application relates generally to a portable aerosol-protective apparatus and method for using the apparatus in a hospital or medical setting.

### BACKGROUND

Healthcare workers are at constant occupational risk for many infectious diseases transmitted from ill patients, despite existing safety protocols. During the recent and severe outbreak of acute respiratory syndrome coronavirus-2 (SARS-CoV-2), many frontline healthcare workers faced a significant risk of contracting the novel coronavirus, despite the use of masks and other personal protective equipment. Although clinical guidelines for the management of patients with acute respiratory diseases and other illnesses exist, the nature and magnitude of the risk encountered by healthcare workers is not well understood.

In general, healthcare workers are most at risk of contracting a respiratory or other illness from a patient during aerosol-generating medical procedures. Procedures that are believed to generate aerosols and droplets as a source of respiratory and other pathogens include positive pressure ventilation (BiPAP and CPAP), high-flow nasal cannula, endotracheal intubation, airway suction, high frequency oscillatory ventilation, tracheostomy, chest physiotherapy, nebulizer treatment, sputum induction, and bronchoscopy, among others. Such procedures are known to stimulate coughing and to promote the generation of aerosols, increasing the risk of infection to healthcare workers.

As demonstrated during the recent novel coronavirus outbreak, a need exists for improved measures to protect healthcare workers from exposure to potentially deadly respiratory pathogens and other illnesses. This need and others are met by the protective apparatus and method described below.

### SUMMARY

Disclosed herein, in one aspect, is a portable aerosol-protective apparatus comprising a plurality of wheels, a height-adjustable support frame that is movably supported on the plurality of wheels, and a shield mounted on the support frame.

The shield can comprise a rear wall defining two openings configured to receive a user's hands, a top wall coupled to and extending forwardly from the rear wall, and a pair of opposing side walls. Each side wall of the pair of opposing side walls can be coupled to and extend forwardly from a respective side of the rear wall and coupled to and extend downwardly from a respective side of the top wall such that the shield forms a partial enclosure defining a receiving space that is sufficient to receive the head-space end of a hospital or surgery bed having a patient lying thereon.

Also disclosed herein is a method of reducing aerosol exposure to a healthcare worker during an aerosol-generating medical procedure, the method comprising performing the medical procedure through the disclosed portable aerosol-protective apparatus.

Additional advantages of the disclosed apparatus and method will be set forth in part in the description which follows, and in part will be understood from the description, or may be learned by practice of the disclosed apparatus and method. The advantages of the disclosed apparatus and method will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed apparatus and method and together with the description, serve to explain the principles of the disclosed apparatus and method.

FIGS. 1A-1D depict several views of an exemplary portable aerosol-protective apparatus. FIG. 1A depicts a side-perspective view, FIG. 1B depicts a rear view, showing the rear wall of the apparatus, and FIGS. 1C and D depict a top and side view, respectively.

FIG. 2 depicts another side-perspective view of the exemplary portable aerosol-protective apparatus.

FIG. 3 is a photograph of another exemplary embodiment of the portable aerosol-protective apparatus partially enclosing the head-space end of a hospital bed, with a healthcare worker's hand extending through one of the openings of the rear wall.

FIG. 4 is a rear perspective view of an exemplary embodiment of the portable aerosol-protective apparatus in a lowered position.

FIG. 5 is a rear perspective view of the exemplary embodiment of FIG. 4 in a raised position.

FIG. 6 is a front perspective view of an exemplary embodiment of the portable aerosol-protective apparatus with protective sleeves attached thereto.

FIG. 7 is a rear perspective view of an exemplary embodiment of the portable aerosol-protective apparatus.

FIG. 8 is a side view of a clip for securing a drape to the portable aerosol-protective apparatus.

FIG. 9 is a rear perspective view of an exemplary embodiment of the portable aerosol-protective apparatus.

FIG. 10 is a schematic diagram of a hardware assembly for allowing a user to initiate raising/lowering of the apparatus.

### DETAILED DESCRIPTION

The disclosed apparatus and method may be understood more readily by reference to the following detailed description of particular embodiments and the examples included therein and to the Figures and their previous and following description.

### A. Definitions

It is to be understood that the terminology used herein is for the purpose of describing particular embodiments only, and is not intended to limit the scope of the present invention which will be limited only by the appended claims.

It must be noted that as used herein and in the appended claims, the singular forms “a,” “an,” and “the” include plural references unless the context clearly dictates otherwise. Thus, for example, reference to “an opening” includes a plurality of such openings, and reference to “the opening” is a reference to one or more openings and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Optionally, in some aspects, when values are approximated by use of the antecedents “about,” “substantially,” or “generally,” it is contemplated that values within up to 15%, up to 10%, up to 5%, or up to 1% (above or below) of the particularly stated value or characteristic can be included within the scope of those aspects.

Unless defined otherwise, all technical and scientific terms used herein have the same meanings as commonly understood by one of skill in the art to which the disclosed appliance and method belong. Although any oral appliance and method similar or equivalent to those described herein can be used in the practice or testing of the present appliance and method, the particularly useful appliances and methods are as described.

Throughout the description and claims of this specification, the word “comprise” and variations of the word, such as “comprising” and “comprises,” means “including but not limited to,” and is not intended to exclude, for example, other elements, components, integers or steps. In particular, in methods stated as comprising one or more steps or operations, it is specifically contemplated that each step comprises what is listed (unless that step includes a limiting term such as “consisting of”), meaning that each step is not intended to exclude, for example, other elements, components, integers or steps that are not listed in the step.

### B. Portable Aerosol-Protective Apparatus

Disclosed herein is an aerosol-protective apparatus that can reduce or eliminate aerosol exposure to a healthcare worker. In general, the aerosol-protective apparatus is portable and can be wheeled from one patient setting to another. In addition, the apparatus can fit around the head-space end of a variety of beds or tables, including hospital and surgery beds. One advantage of the apparatus is that it does not sit on or otherwise touch the patient support structure, in contrast to an intubation box that sits on top of the end of a hospital bed in an unstable manner, posing risks to the patient and medical staff and creating difficulties during a medical procedure.

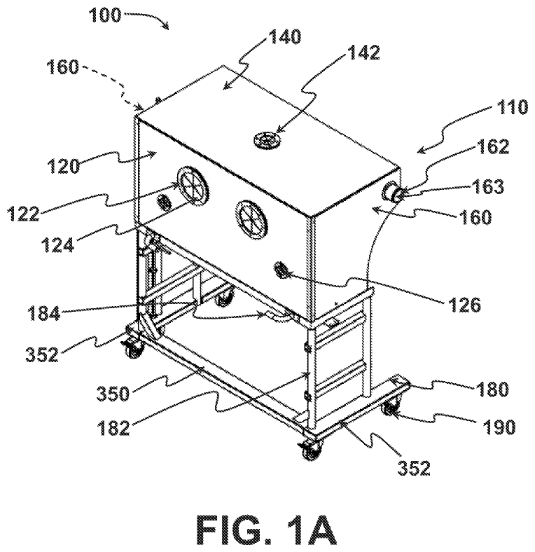

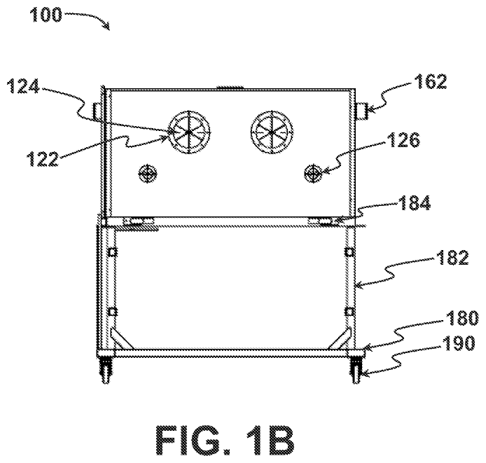

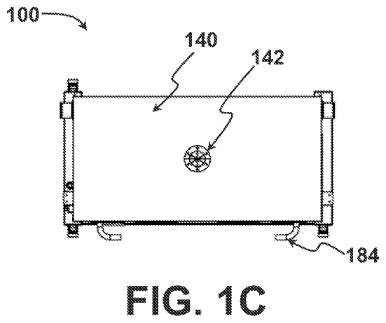

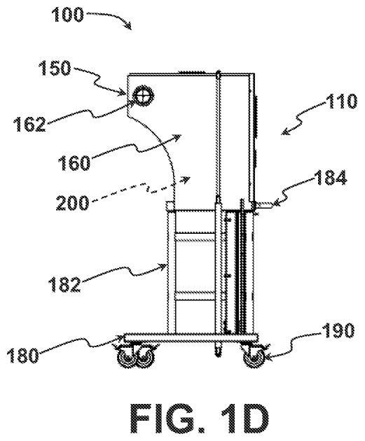

Referring to FIGS. 1A-D, an exemplary portable aerosol-protective apparatus 100 can comprise a shield 110 having a rear wall 120. Rear wall 120 can be substantially or completely transparent, enabling a healthcare worker to easily view a patient during a medical procedure. In addition, rear wall 120 can be substantially rigid or rigid, allowing for the healthcare worker to safely perform a procedure on a patient while standing or sitting behind shield 110.

Rear wall 120 can have a substantially smooth or smooth surface to inhibit contaminants being lodged into (or otherwise engaging) a texture of the wall surface and to facilitate easy cleaning of the wall surface before and after a medical procedure. According to one aspect, rear wall 120 can comprise a transparent and substantially rigid material that is lightweight and shatter-resistant, such as a thermoplastic poly(methyl methacrylate) material (e.g, acrylic glass, Plexiglas, and the like), or other polymer. In some optional aspects, it can be desirable that the material(s) of rear wall 120 are transparent, substantially rigid, without texture, and resistant to contamination, corrosion, peeling, bending, rust, staining, and distortion.

It is contemplated that a healthcare worker can access a patient through at least one of the walls of shield 110, including rear wall 120. Thus, according to one aspect, rear wall 120 can define at least two openings 122 configured to receive a user's hands. In a further aspect, a grommet 124 can be positioned within opening 122. Grommet 124 can define at least one resilient flap configured to resiliently flex from a blocking position to an open position, allowing a healthcare worker to insert a hand into opening 122. Grommet 124 can comprise a flexible material such that the grommet can be capable of being force fitted through opening 122 of rear wall 120.

The diameter of opening 122 can be any diameter suitable for comfortably receiving at least one of a user's hands. To accommodate the hands of most users, opening 122 can have a diameter of at least about six inches. For example, opening 122 can have a diameter of from about six inches to about twelve inches, e.g., from about six to about ten inches, or from about six to about eight inches. Opening 122 can be sufficient to accommodate a grommet that can receive the user's hand.

According to one aspect, rear wall 120 can define at least two openings 122 having a respective flexible sleeve extending forwardly therefrom (i.e., toward the patient). The flexible sleeve can comprise a glove element configured to receive each finger of a user's hand, such that the user can access a patient through the sleeve without allowing air to pass through the respective openings. The flexible sleeve can be made of a material suitable for a medical setting, including for example hypalon, butadyl, neoprene, and butyl, among other materials conventionally used for sleeves and gloves attached to glove boxes.

In addition to openings 122, which can be configured to receive a user's hands, rear wall 120 can optionally define at least one opening 126 (optionally, a plurality of openings) configured to receive therethrough a medical instrument or component thereof. Thus, for example, it is contemplated that at least one opening 126 (optionally, each opening of a plurality of openings 126) can receive a connector or cable of a medical instrument or device during a procedure. Similar to opening 122, opening 126 can also include a grommet positioned within the opening. The grommet can define at least one resilient flap configured to resiliently flex from a blocking position to an open position, allowing for opening 126 to receive a medical instrument or component thereof. The at least one grommet flap can return to the blocking position once the instrument has been inserted into opening 126, and the at least one flap can collapse onto any cable or connector that remains extended through the opening. Opening 126 can have any diameter suitable for receiving a variety of medical instruments or connector or cable thereof. In one aspect, for example, opening 126 can be smaller than opening 122.

Shield 110 of the exemplary apparatus 100 depicted in FIGS. 1A-D also includes a top wall 140 coupled to and extending forwardly from rear wall 120 (i.e., over a portion of the patient). Top wall 140 can be substantially or completely transparent, enabling a healthcare worker to easily view a patient through shield 110 during a medical procedure. In addition, top wall 140 can be substantially rigid or rigid, allowing for the healthcare worker to safely perform a procedure on a patient while standing or sitting behind shield 110.

To minimize contamination, top wall 140 can have a substantially smooth or smooth surface, to avoid contaminants being lodged into (or otherwise engaging) a texture of the wall surface and to facilitate easy cleaning of the wall surface before and after a medical procedure. According to one aspect, top wall 140 can be formed from a transparent and substantially rigid material that is lightweight and shatter-resistant, such as a thermoplastic poly(methyl methacrylate) material (e.g., acrylic glass, Plexiglas, and the like), or other polymer. In some optional aspects, it can be desirable that the material(s) of rear wall 120 are transparent, substantially rigid, without texture, and resistant to contamination, corrosion, peeling, bending, rust, staining, and distortion.

Top wall 140 can optionally define at least one opening 142 (optionally, a plurality of openings 142) configured to receive a medical instrument or component thereof. Thus, for example, it is contemplated that at least one opening 142 (optionally, each opening of a plurality of openings 142) can receive a connector or cable of a medical instrument or device during a procedure. A grommet can be positioned within the opening 142. The grommet can define at least one resilient flap configured to resiliently flex from a blocking position to an open position, allowing for opening 142 to receive therethrough a medical instrument or component thereof. The at least one grommet flap can return to the blocking position once the instrument has been inserted into opening 142, and the at least one flap can collapse onto any cable or connector that remains extended through the opening. Opening 142 can have any diameter suitable for receiving a variety of medical instruments or connector or cable thereof, including for example a line for administering medication to a patient intravenously.

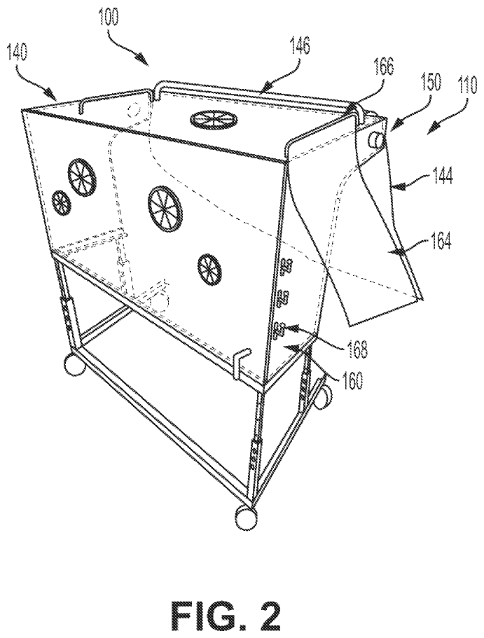

Referring to FIG. 2, shield 110 can optionally comprise a flexible front drape 144 capable of being extended downwardly from a front portion of top wall 140. Flexible front drape 144 can be made of a transparent and flexible material, such as clear vinyl or other materials known in the art, including for example STERI-DRAPE surgical drape material (3M).

When in use, flexible front drape 144 can provide additional protection for a healthcare worker standing or sitting in front of shield 110. In some aspects, flexible front drape 144 can have a length sufficient to cover a portion of the patient's upper body, for example all or part of the patient's chest or abdomen. According to one aspect, as described below, when negative pressure is applied as further described herein, flexible front drape 144 can aid in the isolation of the patient from the surrounding atmosphere by forming at least a partial seal around a portion of the patient's upper body. Thus, a portion of flexible front drape 144 can touch at least a portion of the patient's upper body when the apparatus is in use.

According to one aspect, as depicted in FIG. 1D and FIGS. 2-3, flexible front drape 144 can be affixed to top wall 140 or to an optional front wall 150 that is coupled to and extends downwardly from a forward edge of top wall 140. Thus, for example, flexible front drape 144 can comprise a peel-off adhesive strip on one end such that an end of flexible front drape 144 can be affixed to top wall 140 or optional front wall 150 extending downwardly from a forward edge of top wall 140.

According to another aspect, top wall 140 can comprise one or more rods 146 to which flexible front drape 144 can be looped around or affixed to, e.g., through an adhesive strip, such that the drape can then be extended downwardly from top wall 140 toward a patient. Similarly, top wall 140 can comprise one or more side rods 166 to which a flexible side drape can be attached, as described in more detail below. In other optional aspects, it is contemplated that a single, continuous rod can extend along the front and side portions of the periphery of the top wall 140. In still further optional aspects, it is contemplated that a single flexible drape 144 can be attached to and extend along the rod or rods that are positioned along the front and side portions of the top wall 140.

Referring again to FIGS. 1A-D, shield 110 further comprises a pair of opposing side walls 160. Each side wall 160 can be coupled to and extend forwardly from a respective side of rear wall 120 and coupled to and extend downwardly from a respective side of the top wall 140. Optionally, each side wall 160 can be integrally formed with the top wall 140 and/or the rear wall 120. According to one aspect, top wall 140 extends farther from rear wall 120 than at least a lower portion of side walls 160. Thus, for example, side walls 160 can include an upper portion that extends the same or about the same distance from rear wall 120 and a lower portion that does not extend as far as the upper portion. Thus, the upper portion of at least one (optionally, each) side wall can have a first maximum length (in a forward direction moving away from the rear wall) that is substantially the same as a length of the top wall, and the lower portion of at least one (optionally, each) side wall can have a second maximum length that is less than the first maximum length such that the lower portion of the side wall is recessed from the front edge of the upper portion of the side wall. In one aspect, side walls 160 can be L-shaped or substantially L-shaped. In other optional aspects, it is contemplated that one or more of the side walls can have a tapered or curved profile having a progressively decreasing length (measured in a forward direction moving away from the rear wall) as the side wall extends downwardly from the top wall. In use, it is contemplated that the varying lengths of each side wall can provide improved access to the patient (through the void areas defined by the portions having reduced length) while also providing sufficient coverage to minimize unnecessary exposure to the patient.

Side walls 160 can be substantially or completely transparent, enabling a healthcare worker to easily view a patient through shield 110 during a medical procedure. In addition, side walls 160 can be substantially rigid or rigid, allowing for the healthcare worker to safely perform a procedure on a patient while standing or sitting behind shield 110. To minimize contamination, side walls 160 can have a substantially smooth or smooth surfaces to avoid contaminants being lodged into (or otherwise engaging) a texture of the wall surface and to facilitate easy cleaning of the wall surface before and after a medical procedure. According to one aspect, side walls 160 can comprise a transparent and substantially rigid material that is lightweight and shatter-resistant, such as a thermoplastic poly(methyl methacrylate) material (e.g., acrylic glass, Plexiglas, and the like), or other polymer. In some optional aspects, it can be desirable that the material(s) of side walls 160 are transparent, substantially rigid, without texture, and resistant to contamination, corrosion, peeling, bending, rust, staining, and distortion.

Optionally, one or both of side walls 160 can define an opening 162 configured to receive negative pressure. Thus, a source of negative pressure can be coupled to opening 162. The source of negative pressure can be useful for arresting aerosol droplets and other contaminants that may be ejected from a patient during a medical procedure. In one aspect, to ensure that any arrested contaminants do not compromise the surrounding atmosphere, the source of negative pressure coupled to opening 162 can be further coupled to a filter having a suitable filter or mesh size such that any contaminant or aerosol droplet can be isolated from the atmosphere. Suitable filters include high-efficiency particulate air (HEPA) filters, which are capable filtering contaminants and aerosol droplets having a diameter of about 0.3 microns or larger.

Although described herein as being provided through openings 162 in the side walls, it is contemplated that negative pressure can additionally, or alternatively, be provided through openings in the top and/or rear walls. Similarly, although described herein as being received through openings in the rear wall, it is contemplated that medical devices and/or surgical tools can be received through openings in the side and/or top walls.

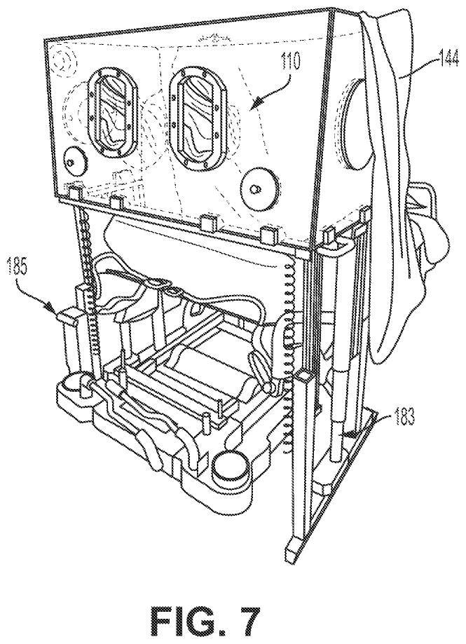

Referring again to FIG. 2, according to one aspect, shield 110 can comprise a flexible side drape 164 capable of being extended downwardly from an upper portion of at least one side wall 160. Flexible side drape 164 can be made of a transparent and flexible material, such as clear vinyl. Side drape 164 can cover any void left by a portion of a side wall 160 that does not extend forwardly the entire distance of top wall 140. Side drape 164 can be affixed to top wall 140 or an upper portion of side wall 160, for example through an adhesive strip as described above. Similarly, side drape 164 can be looped around or otherwise affixed to a rod 166, which can be mounted to top wall 140 as shown in FIG. 2 or alternatively mounted to an upper portion of side wall 160. Although described herein as a separate element from front drape, it is contemplated that the side drapes 164 and the front drape can be provided as a single drape structure. For example, as shown in FIG. 7, the front drape 144 can extend across the entire front of the shield 110 and fall over the side walls 160 of the shield.

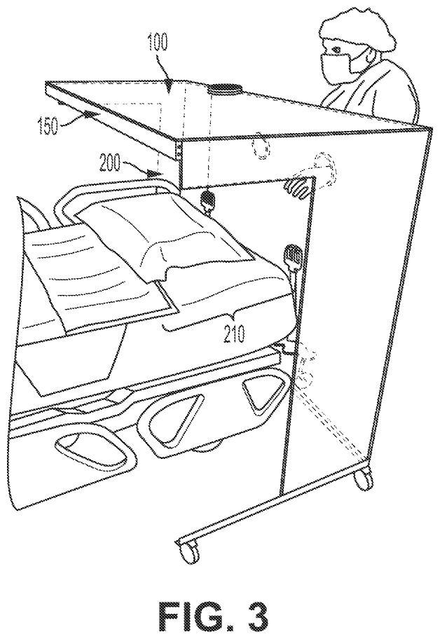

As shown in FIG. 1D and FIG. 3, shield 100 forms a partial enclosure 200 defining a receiving space that is sufficient to receive the head-space end 210 of a hospital or surgery bed having a patient lying thereon. Thus, in contrast to an isolation device in which the entire patient is isolated from the surrounding atmosphere, apparatus 100 allows for a healthcare worker to easily access the patient while simultaneously protecting the professional from contaminants such as aerosol droplets.

In general, partial enclosure 200 defined by shield 100 can have any dimension suitable for receiving the head-space end 210 of a hospital or surgery bed having a patient lying thereon. The dimensions can vary depending on the size of the patient and the bed. In general, the depth of partial enclosure 200 (defined by at least the depth of top wall 140 shown in FIG. 1A) can be sufficient to cover at least the head of the patient, including for example the head and neck of the patient, and can extend below the neck according to one aspect. The width of partial enclosure 200 can in general be sufficient to fit around the head of a hospital or surgery bed. Hospital or surgery beds typically range from about 20 inches to about 60 inches in width.

Similarly, the height of partial enclosure 200 can be sufficient to form a canopy over a patient lying in a hospital or surgery bed in any position or configuration. Thus, for example, the height of partial enclosure 200 can be such that the patient would not bump into the top wall of shield 110 if the patient were to raise from a lying position. Partial enclosure 200 can have a height sufficient for accommodating a patient in any position or configuration, including for example, the fowler's position, lateral position, lithotomy position, prone position, supine position, Trendelenburg or reverse Trendelenburg position, and sim's position, among others. The height of partial enclosure 200 can also be adjusted as described below through a support frame, which allows shield 110 to accommodate a variety of hospital and surgery beds with adjustable heights.

Referring again to FIG. 1D and FIGS. 2-3, shield 110 can optionally comprise a front wall 150 that is coupled to and extends downwardly from a forward edge of top wall 140. Like top wall 140 and side walls 160, optional front wall 150 can be substantially or completely transparent. In addition, optional front wall 150 can be substantially rigid or rigid. According to one aspect, as depicted in FIG. 1D and FIGS. 2-3, optional front wall 150 can be a partial wall that extends downwardly from top wall 140 but does not extend downwardly as far as the side and/or rear walls.

Optional front wall 150 can have a substantially smooth or smooth surface, to avoid contaminants being lodged into (or otherwise engaging) a texture of the wall surface and to facilitate easy cleaning of the wall surface before and after a medical procedure. According to one aspect, optional front wall 150 can comprise a transparent and substantially rigid material that is lightweight and shatter-resistant, such as a thermoplastic poly(methyl methacrylate) material, including for example acrylic glass, Plexiglas, and the like, or other polymer. In some optional aspects, it can be desirable that the material(s) of optional front wall 150 are transparent, substantially rigid, without texture, and resistant to contamination, corrosion, peeling, bending, rust, staining, and distortion.

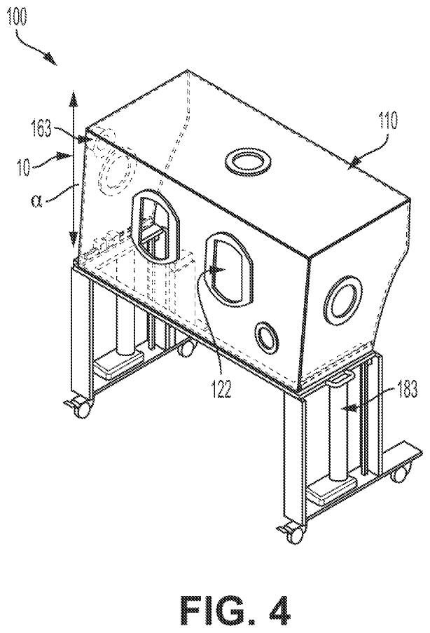

As depicted in FIGS. 1A-D, exemplary apparatus 100 comprises a shield 110 mounted on support frame 180. According to one aspect, support frame 180 can be height-adjustable. For example, support frame 180 can comprise height-adjustable legs 182 that are movable about and between a collapsed configuration and a fully extended configuration. Optionally, the height-adjustable legs 182 can be positioned in at least one additional position (optionally, a plurality of additional positions) between the collapsed configuration and the fully extended configuration. In some exemplary aspects, it is contemplated that the height-adjustable legs 182 can comprise a base portion and an upper portion that is telescopically coupled to the base portion. In additional aspects, it is contemplated that height-adjustable legs 182 can be spring-biased toward an extended configuration or can be extended through a hydraulic or pressurized piston mechanism. Thus, height-adjustable legs 182 can be raised or lowered via a push button or other user interface, which can be positioned in proximity to the apparatus 100, for example, on support frame 180. More generally, it is contemplated that any mechanical, hydraulic, and/or electrical actuator 183 (FIG. 4) that permits selective adjustment of an operative length of each leg 182 can be employed. It is contemplated that such actuators (e.g., electrical actuators) can provide a quick, safe, and easy height adjustment. Alternatively, it is contemplated that height-adjustable legs 182 can be adjusted manually, for example using locking pins as are known in the art.

Support frame 180 can be movably supported on a plurality of wheels 190. According to one aspect. the plurality of wheels 190 can be casters. The casters can be lockable to keep apparatus 100 in place while in use and freely swivelable (freely able to swivel) and movable when in an unlocked position, enabling apparatus 100 to be easily moved from one patient setting to another. According to one aspect, the plurality of wheels 190 can include two front wheels and two rear wheels, all of which can be lockable casters.

The portable aerosol-protective apparatus can optionally comprise a variety of other features that can be advantageous or provide convenience in a medical setting. In one aspect, for example, as depicted in FIGS. 1A-D, support frame 180 can comprise one or more handles 184 for moving apparatus 100. In addition, with reference to FIG. 2, one or more side walls 160 can comprise one or more hooks 168 or other holders for accommodating medical equipment or for another convenient use.

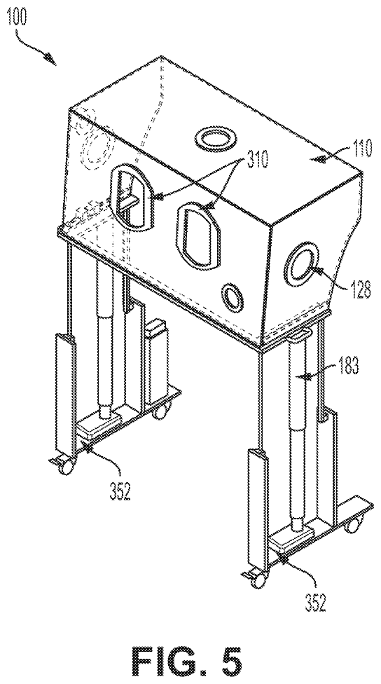

Referring to FIGS. 4-7, is an embodiment of an exemplary apparatus 100 that can advantageously be used for intubation. The openings 122 can advantageously be elongate relative to a vertical axis 10. For example, the openings 122 can have a major dimension relative to the vertical axis 10 that is at least two inches greater than a minor dimension relative to a horizontal axis. In some optional aspects, the openings 122 can have a major dimension of about 6 inches to about 12 inches (preferably, about 10 inches) and a minor dimension of about 3 inches to about 9 inches (preferably, about 5 inches. It is contemplated that such openings 122 that are elongate relative to the vertical axis 10 can promote and permit movements that are used during intubation (e.g., downward thrusting movement). Optionally the longitudinal ends of the openings can be arcuate.

In some aspects, sleeves 300 can be coupled to the shield 110 at one or each opening 122. The sleeves 300 can receive a clinicians arms therein and allow the clinician to move her arms while inhibiting air from the partial enclosure 200 escaping through the openings 122. Accordingly, the sleeves 300 can be flexible. For example, sleeves 300 can comprise flexible polymer (optionally, transparent polymer). Each sleeve 300 can have a proximal end that couples to the shield 110 and an opposed distal end 304. The distal end 304 can define an opening 308 to receive a clinician's hand therethrough. The distal end 304 can comprise elastic 306 to constrict the area of the opening of the distal end to retract the distal end around the arm of the clinician. In some optional aspects, the sleeve can have a length that is sufficient to receive an entire arm of a clinician (e.g., at least 15, at least 20, or at least 25 inches) so that the opening 308 of the distal end 304 is positioned about the wrist or forearm of the clinician. It is contemplated that the openings can be positioned to engage a glove worn on the clinician's hand so that no portion of the clinician's hand or arm is exposed to the interior of the partial enclosure 200.

Optionally, fastening rings 310 can couple the sleeves 300 to the shield 110. For example, each fastening ring 310 can extend around the circumference of the respective openings 122 and can be secured against the shield 110 (e.g., via fasteners, such as screws, etc.) with the proximal end 302 of the sleeve 300 compressed between the shield 110 and the fastening ring 310 to retain the sleeve 300 therebetween. In further aspects, each ring 310 and respective sleeve 300 can be coupled together (optionally, integrally formed). The rings can couple to the shield 110 (optionally, via snaps or other quick-release fasteners to allow rapid attachment and/or removal), thereby coupling the sleeve to the shield. In some optional aspects, a gasket can be positioned between the shield and each ring 310 to form an airtight seal between the shield and the ring. It is contemplated that the sleeves 300 can be removed, sanitized, and reused.

One or both of the side walls 160 can define at least one opening 128 that can enable an assistant (e.g., another clinician) to reach therethrough. When not in use, each opening 128 can receive a respective cover 129 that inhibits air travel through the opening 128. Optionally, a sleeve 300 or a grommet 124 can be positioned within the opening 128.

A tubular fitting 163 can be positioned at the opening 162 for coupling to a conduit (e.g., hose) of a negative pressure source. In some optional aspects, the tubular fitting can have a diameter of less than four inches (e.g., about 3.2 inches). In some aspects, it is contemplated that the apparatus 100 can comprise only one opening 162 (and thus, only a single filter). In this way, the vacuum drawn from air being pulled through the opening 162 can be of sufficient but not excessive negative pressure.

The opening 162 can be positioned toward the upper rear of corner of the side wall 160. That is, the opening 162 can be closer to the rear wall 120 than to the forward edge of the side wall 160, and the opening 162 can be closer to the top wall 140 than the bottom edge of the side wall. In this way, it is contemplated that aerosol droplets can most effectively be removed from the partial enclosure 200. For example, the apparatus 100 can be positioned so that head of the patient is positioned toward the rear of the partial enclosure 200. Accordingly, as air is drawn out of the partial enclosure 200 through the opening 162, clean air can be drawn in from underneath the flexible front drape 144 and across the head of the patient toward the opening 162. Thus, the position of the opening 162 can create minimal turbulent air flow, thereby safely and optimally providing exchange air within the partial enclosure 200. It is further contemplated that the positioning of the opening 162 can direct all tubing through the side wall 160 and avoid interference with the hands of a clinician accessing the patient through the rear wall 120.

In some aspects, the rear wall 120 can be forwardly angled relative to a vertical plane that extends parallel to the length of the apparatus 100. For example, the lower edge of the rear wall 120 can be rearwardly positioned relative to the upper edge of the rear wall. Optionally, the rear wall 120 can form an angle, α, with a vertical axis that is at least two degrees, between two degrees and 10 degrees, or about 5 degrees. It is contemplated that this angle can enhance the ergonomics of the shield by allowing the clinician to place his or her arms through the openings 122 at a natural downward angle.

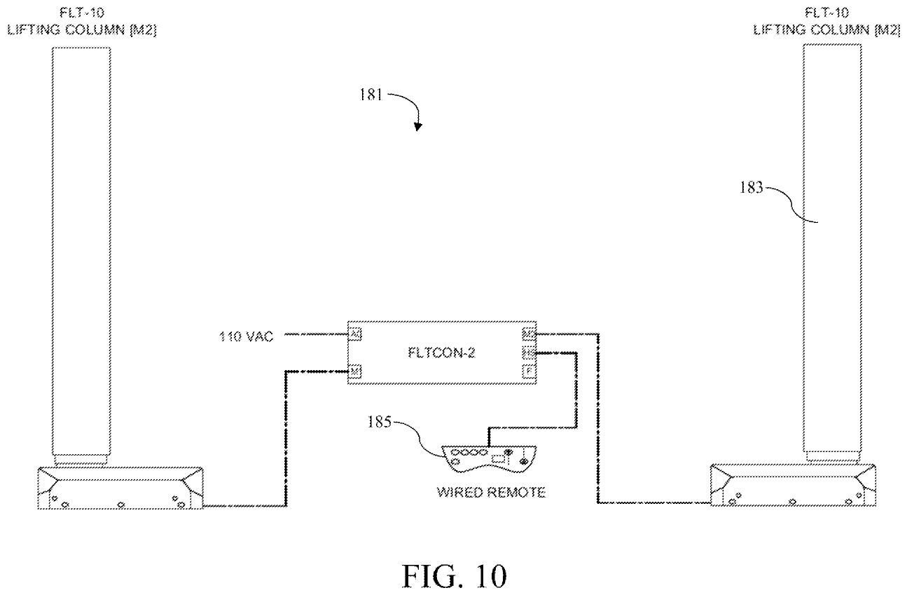

In some aspects, the apparatus 100 can comprise a memory that stores one or more predetermined shield heights (e.g., four predetermined shield heights). For example, in various clinical environments, bed heights can be standardized. Accordingly, a user can select the predetermined shield height depending on the bed in which the patient is positioned. In further aspects, the predetermined shield height(s) can be determined based on the user. For example, a tall user can have one or more relatively higher predetermined height settings, and a shorter user can have one or more relatively lower predetermined height settings. As shown in FIG. 10, the apparatus can comprise an input device 185 (e.g., a plurality of buttons corresponding to different predetermined heights, a smart phone, or other computing device having a user interface or capable of receiving an input from a user). A user can use the input device to select a predetermined height, and the actuator 183 can move to a predetermined position, thereby positioning the shield 110 the corresponding predetermined height. Optionally, the set shield heights can be standardized. In further aspects, the input device can enable a user to manually adjust the height of the shield. In still further aspects, the input device can enable the user to save a certain height. In still further aspects, the input device can comprise a keypad that enables the user to input a particular desired shield height. For example, the input device can comprise a numerical keypad. In this way, a user can, for example, input a height in inches or centimeters. In various optional aspects, the input device can comprise a knob, a switch, a plurality of switches, a keypad, or any other suitable device. In one optional aspect, the input device can comprise an up momentary switch, a down momentary switch, and four momentary switches that correspond to saved predetermined positions. Optionally, the actuator 183 and input device can comprise a FLT-10 Lifting Column Set by Progressive Automations Inc. (Arlington, Wash.). In various further embodiments, the input device can comprise a smartphone, tablet, or other computing device in communication with the actuator, a microphone in communication with a voice-controlled system, a keyboard, touchscreen display, pointing device (e.g., a computer mouse, remote control), a joystick, a scanner, tactile input devices such as gloves, and other body coverings, motion sensor, and the like.

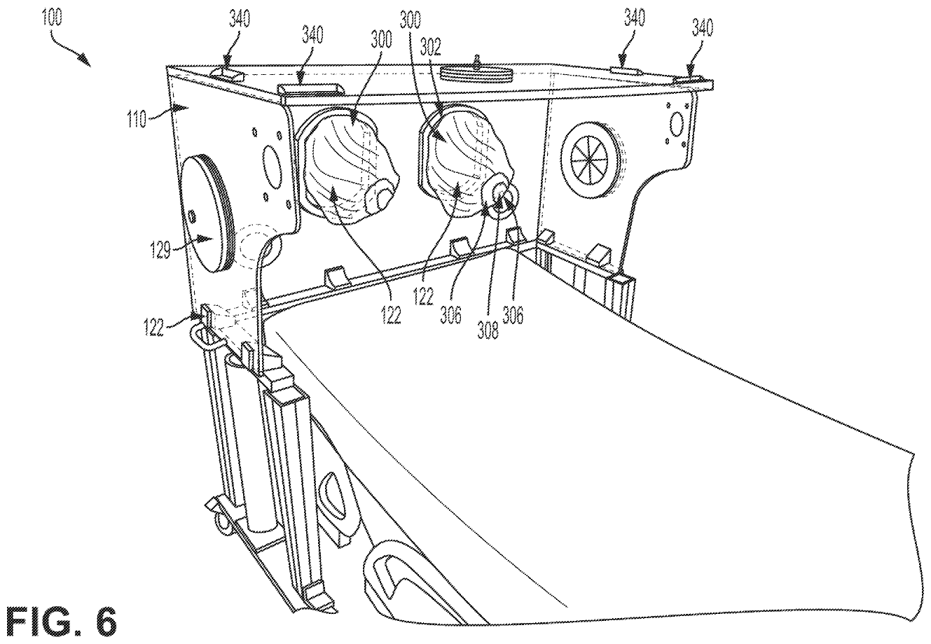

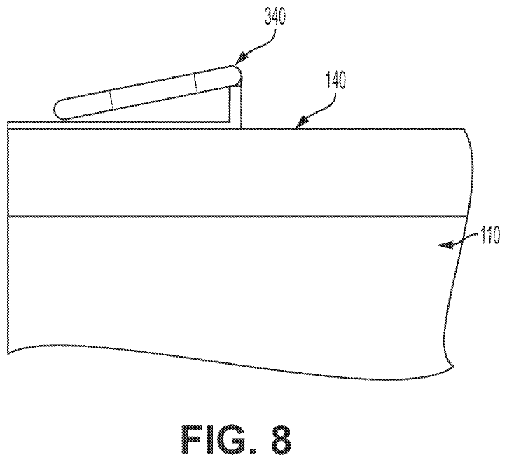

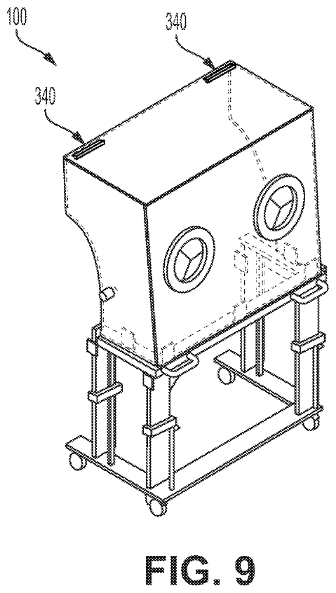

Referring to FIGS. 7-9, the apparatus 100 can comprise one or more retainer clips 340 that affix the flexible front drape 144 to the shield 110. The retainer clips 340 can be rotationally biased (optionally, via a torsion spring) to bias against a surface (e.g., a top surface) of the shield 110. In some aspects, the apparatus 100 can comprise at least one retainer clip 340 on the top wall 140 proximate to the front edge of the top wall 140 on each side of the shield. In further aspects, the apparatus 100 can comprise at least one retainer clip on the top wall 140 proximate to each side wall 160 and rearwardly spaced from the front wall. Accordingly, in some aspects, the apparatus 100 can comprise two (FIG. 9) or four (FIG. 6) retainer clips 340. Optionally, the retainer clips 340 can be elongated along the edge of the top wall proximate to which the retainer clips are positioned.

In some aspects, the support frame 180 can comprise a cross beam 350 (FIG. 1) that extends between rear portions of both sides 352 of the support frame. Optionally, the support frame 180 can omit the cross beam 350 so that no cross member extends between the sides 352 of the support frame below a height of 24 inches or below a height of 36 inches, thereby omitting any support member that can inhibit positioning of the clinician's legs when using the apparatus 100.

FIG. 9 illustrates an apparatus 100 that can be configured for an operating room. For example, it can optionally have a narrower width (e.g., 28 inches between the opposing side walls) than the apparatus can be configured for the operating room and can omit any openings on the side walls. In some aspects, the apparatus 100 for operating room usage can provide only two access points (i.e., the openings 122 through the rear wall). In further aspects, it is contemplated that the longitudinal dimension of the apparatus 100, when used for operating room purposes, can be reduced in comparison to an apparatus 100 for intubation, since an operating room patient will generally remain flat during use.

In various aspects, the shield 110 can comprise polycarbonate or other suitable polymer. In some aspects, the drape 144 can comprise polyvinylchloride PVC or other suitable polymer.

FIG. 10 illustrates an exemplary, optional embodiment of a hardware assembly 181 for raising and lowering the shield 110. The hardware assembly can comprise a pair of actuators 183 coupled to an input device 185. The input device 185 can comprise a wired or wireless remote having up and down buttons that raise and lower, respectively, the actuators 183 when pressed. The up and down buttons can be momentary buttons that, when held, can cause the actuators 183 to move, and, when released can cause the actuators 183 to cease movement. To set a preset location, the shield can be moved to a desired height. A button (e.g., designated “M”) can be pressed, followed by a numbered button (e.g., 1-4), to set the corresponding numbered button to the current height. The input device 185 can comprise a display (e.g., an LED display) that displays an indication (e.g., an “S” followed by the pressed number) to indicate that the height has been saved. To use a preset height, the user can press the numbered button, and the actuators can move until the shield 110 reaches the preset height. The display can further display error codes indicating corresponding errors.

In further aspects, the height can be numerically set. For example, one or more numbered buttons can be set to correspond to a desired height. In further aspects, the shield can be set to a starting height. For example, the user can press and hold the “M” button until the display flashes an indicator (e.g., “RST”). The user can then press the up and down buttons to select a particular height, as displayed on the display.

The hardware assembly 181 can comprise one or more sensors for determining one or more states of the hardware assembly. In various aspects, the hardware assembly 181 can comprise limit switches for inhibiting movement of the actuators 183 beyond minimum or maximum thresholds. Optionally, the limit switches can comprise, for example, Hall Effect sensors. In further aspects, the hardware assembly 181 can comprise current sensors that are configured to detect overcurrent or insufficient current. In still further aspects, the hardware assembly 181 can comprise at least one temperature sensor for detecting overheating. It is contemplated that exceeding thresholds of the one or more sensors can cause the display to display an output corresponding to the surpassing of the given threshold, thereby providing a status for enabling troubleshooting. In some optional aspects, the hardware assembly 181 can have a low power mode to reduce power consumption after inactivity for a predetermined duration.

### C. Method for Reducing Aerosol Exposure to a Healthcare Worker

Also disclosed herein is a method of reducing aerosol exposure to a healthcare worker during an aerosol-generating medical procedure, the method comprising performing the medical procedure through the portable aerosol-protective apparatus described above. The method is suitable for a variety of aerosol-generating medical procedures in which respiratory and other pathogens may be generated, including without limitation high-flow nasal cannula, positive pressure ventilation (BiPAP and CPAP), endotracheal intubation, airway suction, high frequency oscillatory ventilation, tracheostomy, chest physiotherapy, nebulizer treatment, sputum induction, and bronchoscopy.

With reference to FIG. 1D and FIG. 3, according to one aspect, the method comprises positioning apparatus 100 such that partial enclosure 200 fits around at least the head-space end of a hospital or surgery bed having a patient thereon, such that the head, neck, and/or chest of the patient is within partial enclosure 200. With reference to FIG. 1A and FIG. 3, once the head-space end of the hospital or surgery bed is within partial enclosure 200, a healthcare worker can insert his or her hands into openings 122 and perform the procedure behind shield 110. Optionally, as described above with reference to FIG. 3, flexible front drape 144 can be extended down across at least a portion of the patient's upper body to further isolate the patient from the surrounding atmosphere.

In a further aspect, with reference to FIG. 1A, negative pressure can be applied to the opening defined by a least one of side walls 160 during at least a portion of the medical procedure. As described above, the negative pressure source can be coupled to a suitable filter for removing aerosols and other contaminants from the atmosphere, such as a HEPA filter.

### Exemplary Aspects

In view of the described products, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.

Aspect 1: A portable aerosol-protective apparatus comprising: a plurality of wheels; a height-adjustable support frame that is movably supported on the plurality of wheels; and a shield mounted on the support frame, the shield comprising: a rear wall defining two openings configured to receive a user's hands: a top wall coupled to and extending forwardly from the rear wall; and a pair of opposing side walls, wherein each side wall of the pair of opposing side walls is coupled to and extends forwardly from a respective side of the rear wall, wherein each side wall of the pair of opposing side walls is coupled to and extends downwardly from a respective side of the top wall; wherein the shield forms a partial enclosure defining a receiving space that is sufficient to receive the head-space end of a hospital or surgery bed having a patient lying thereon.

Aspect 2: The apparatus of aspect 1, wherein the top wall extends farther, in a forward direction, from the rear wall than at least a lower portion of the side walls.

Aspect 3: The apparatus of aspect 2, wherein the side walls are substantially L-shaped.

Aspect 4: The apparatus of any one of the preceding aspects, further comprising a flexible front drape capable of being extended downwardly from a front portion of the top wall.

Aspect 5: The apparatus of any one of the preceding aspects, further comprising a flexible side drape capable of being extended downwardly from an upper portion of at least one side wall.

Aspect 6: The apparatus of any one of the preceding aspects, wherein at least one of the side walls defines an opening configured to receive negative pressure.

Aspect 7: The apparatus of any one of the preceding aspects, wherein each side wall defines an opening configured to receive negative pressure.

Aspect 8: The apparatus of any one of the preceding aspects, wherein the top wall defines at least one opening configured to receive a medical instrument or component thereof.

Aspect 9: The apparatus of any one of the preceding aspects, wherein the rear wall defines at least one opening configured to receive a medical instrument or component thereof.

Aspect 10: The apparatus of any one of the preceding aspects, wherein the plurality of wheels are lockable casters.

Aspect 11: The apparatus of any one of the preceding claims, wherein the support frame comprises height-adjustable legs.

Aspect 12: The apparatus of aspect 11, wherein the height-adjustable legs are movable about and between a collapsed configuration and an extended configuration and are spring-biased toward an extended configuration.

Aspect 13: The apparatus of any one of the preceding aspects, wherein the rear, top, and side walls are substantially rigid and transparent.

Aspect 14: The apparatus of any one of the preceding aspects, wherein the rear, top, and side walls have substantially smooth surfaces.

Aspect 15: The apparatus of any one of the preceding aspects, further comprising a grommet positioned within each opening of the at least two openings, wherein each grommet defines at least one resilient flap that is configured to resiliently flex from a blocking position to an open position.

Aspect 16: The apparatus of any one of the preceding aspects, wherein the shield further comprises a front wall that is coupled to and extends downwardly from a forward edge of the top wall.

Aspect 17: A method of reducing aerosol exposure to a healthcare worker during an aerosol-generating medical procedure, the method comprising performing the medical procedure through the portable aerosol-protective apparatus of any of aspects 1-16 or aspects 21-29.

Aspect 18: The method of aspect 17, wherein the medical procedure is performed through the portable aerosol-protective apparatus of claim 6 or claim 7, and wherein negative pressure is applied to the opening defined by at least one of the side walls during at least a portion of the procedure.

Aspect 19: The method of aspect 17, wherein the medical procedure comprises intubating or extubating a patient.

Aspect 20: The method of aspect 17, wherein the medical procedure comprises a bronchoscopy or the use of a device selected from a high-flow nasal cannula, continuous positive airway pressure (CPAP) device, or a bilevel positive airway pressure (BiPAP) device.

Aspect 21: The apparatus of any one of aspects 1-16, wherein the two openings of the rear wall are elongate relative to a vertical axis.

Aspect 22: The apparatus of any one of aspects 6-16, wherein the opening defined by the at least one side wall is configured for coupling to a negative pressure source, wherein the opening is positioned closer to the top wall than a lower edge of the at least one side wall.

Aspect 23: The apparatus of aspect 22, wherein a tubular fitting is positioned at the opening of the at least one sidewall.

Aspect 24: The apparatus of aspect 22 or aspect 23, wherein the opening defined by the at least one side wall is positioned closer to the rear wall than a forward edge of the at least one side wall.

Aspect 25: The apparatus of any one of aspects 1-16 or 21-24, wherein the rear wall has a lower edge and an upper edge, wherein the rear wall is angled relative to a vertical axis by at least 2 degrees so that the lower edge is rearwardly positioned relative to the upper edge.

Aspect 26: The apparatus of any one of aspects 1-16 or 21-25, further comprising a sleeve positioned over each of the two openings defined by the rear wall, wherein the sleeve comprises a proximal end and a distal end, wherein the proximal end is coupled to the shield, wherein the distal end defines an opening having an area, wherein the sleeve further comprises elastic that is configured to constrict the area of the opening of the distal end.

Aspect 27: The apparatus of any one of aspects 1-16 or 21-26, wherein the at least one sidewall further defines at least one arm opening that is configured to receive an arm of a clinician therethrough.

Aspect 28: The apparatus of any one of aspects 1-16 or 21-27, further comprising: an actuator that is configured to adjust a height of the shield; a memory that is configured to store an instruction corresponding to at least one actuator position; and an input device that is configured to receive an input from a user and, in response. cause the actuator to move to the at least one actuator position.

Aspect 29: The apparatus of any one of aspects 1-16 or 21-28, further comprising at least one clip that is configured to secure a drape to the shield.

Those skilled in the art will recognize, or be able to ascertain using no more than routine experimentation, many equivalents to the specific embodiments of the apparatus and method described herein. Such equivalents are intended to be encompassed by the following claims.

1. A portable aerosol-protective apparatus comprising:
a) a plurality of wheels;
b) a height-adjustable support frame that is movably supported on the plurality of wheels; and
c) a shield mounted on the support frame, the shield comprising:
a rear wall defining two openings configured to receive a user's hands;
a top wall coupled to and extending forwardly from the rear wall; and
a pair of opposing side walls, wherein each side wall of the pair of opposing side walls is coupled to and extends forwardly from a respective side of the rear wall, wherein each side wall of the pair of opposing side walls is coupled to and extends downwardly from a respective side of the top wall;
wherein the shield forms a partial enclosure defining a receiving space that is sufficient to receive the head-space end of a hospital or surgery bed having a patient lying thereon.

2. The apparatus of claim 1, wherein the top wall extends farther, in a forward direction, from the rear wall than at least a lower portion of the side walls.

3. The apparatus of claim 2, wherein the side walls are substantially L-shaped.

4. The apparatus of claim 1, further comprising a flexible front drape capable of being extended downwardly from a front portion of the top wall or from an upper portion of at least one side wall.

5. (canceled)

6. The apparatus of claim 1, wherein at least one of the side walls defines an opening configured to receive negative pressure.

7. (canceled)

8. The apparatus of claim 1, wherein the top wall defines at least one opening configured to receive a medical instrument or component thereof.

9. The apparatus of claim 1, wherein the rear wall defines at least one opening configured to receive a medical instrument or component thereof.

10. (canceled)

11. The apparatus of claim 1, wherein the support frame comprises height-adjustable legs, wherein the height-adjustable legs are movable about and between a collapsed configuration and an extended configuration and are spring-biased toward an extended configuration.

12. (canceled)

13. The apparatus of claim 1, wherein the rear, top, and side walls are substantially rigid and transparent.

14. (canceled)

15. The apparatus of claim 1, further comprising a grommet positioned within each opening of the at least two openings, wherein each grommet defines at least one resilient flap that is configured to resiliently flex from a blocking position to an open position.

16. The apparatus of claim 1, wherein the shield further comprises a front wall that is coupled to and extends downwardly from a forward edge of the top wall.

17. The apparatus of claim 1, wherein the two openings of the rear wall are elongate relative to a vertical axis.

18. The apparatus of claim 6, wherein the opening defined by the at least one side wall is configured for coupling to a negative pressure source, wherein the opening is positioned closer to the top wall than a lower edge of the at least one side wall.

19. The apparatus of claim 18, wherein a tubular fitting is positioned at the opening of the at least one side wall.

20. The apparatus of claim 18, wherein the opening defined by the at least one side wall is positioned closer to the rear wall than a forward edge of the at least one side wall.

21. The apparatus of claim 1, wherein the rear wall has a lower edge and an upper edge, wherein the rear wall is angled relative to a vertical axis by at least 2 degrees so that the lower edge is rearwardly positioned relative to the upper edge.

22. The apparatus of claim 1, further comprising a sleeve positioned over each of the two openings defined by the rear wall, wherein the sleeve comprises a proximal end and a distal end, wherein the proximal end is coupled to the shield, wherein the distal end defines an opening having an area, wherein the sleeve further comprises elastic that is configured to constrict the area of the opening of the distal end.

23. The apparatus of claim 1, wherein the at least one sidewall further defines at least one arm opening that is configured to receive an arm of a clinician therethrough.

24. The apparatus of claim 1, further comprising:
an actuator that is configured to adjust a height of the shield;
a memory that is configured to store an instruction corresponding to at least one actuator position; and
an input device that is configured to receive an input from a user and, in response, cause the actuator to move to the at least one actuator position.

25. (canceled)

26. A method of reducing aerosol exposure to a healthcare worker during an aerosol-generating medical procedure, the method comprising performing the medical procedure through the portable aerosol-protective apparatus of claim 1.

27. (canceled)

28. (canceled)

29. (canceled)
